(19) United States
(12) Patent Application Publication
(10) Pub. No.: US 2015/0285803 A1
(43) Pub. Date: Oct. 8, 2015

(54) SYSTEMS AND METHODS FOR IDENTIFYING PROGESTERONE RECEPTOR SUBTYPES

(71) Applicants: Erard Gilles, Bridgewater, NJ (US); Alexander Zukiwski, Clarksburg, MD (US)

(72) Inventors: Erard Gilles, Bridgewater, NJ (US); Alexander Zukiwski, Clarksburg, MD (US)

(73) Assignees: INVIVIS PHARMACEUTICALS INC., Bridgewater, NJ (US); ARNO THERAPEUTICS, INC., Flemington, NJ (US)

(21) Appl. No.: 14/681,032

(22) Filed: Apr. 7, 2015

Related U.S. Application Data
(60) Provisional application No. 61/976,872, filed on Apr. 8, 2014.

(51) Int. Cl. G01N 33/574

(52) U.S. Cl. CPC G01N 33/57415; G01N 2333/723

(57) ABSTRACT

Systems and methods for identifying progesterone positive tumors utilizing progesterone receptor isotype-specific antibodies are provided.

This application claims priority to U.S. Provisional Patent Application Ser. No. 61/976,872, filed Apr. 8, 2014. The above referenced application is incorporated herein by reference as if restated in full.

### BACKGROUND

Triple negative breast cancer (TNBC) refers to breast cancer (BC) tumors that do not express the estrogen receptor (e.g., estrogen receptor α (ER α)), progesterone receptor (PR), and HER2/neu. Patients with TNBC have limited to response to current treatments for BC (e.g., hormonal treatment and anti-HER2 treatment). Given the limited therapeutic options for TNBC, diagnostic accuracy is an important consideration in determining the appropriate treatment for a patient. PR has been used as a surrogate marker for estrogen receptor (ER), indicating the presence of undetected ER. Current diagnostic tools have not been directed at PR as a target for direct therapeutic treatment.

The progesterone receptor (PR) belongs to the steroid receptor family, a subset of the nuclear receptor family and is the mediator of progesterone action. PR is a ligand-activated transcription factor which plays a key role in hormonally regulated tissues and is expressed in the human as two major forms; progesterone receptor A (PRA) and progesterone receptor B (PRB). In routine immunohistochemistry (IHC) testing of breast cancer tissue specimens, bispecific antibodies (Ab) that recognize epitopes common to both PR isotypes (e.g., PR subtype A (PRA) and PR subtype B (PRB)) are used to determine the presence of the PR. Thus, determining PR status using current clinical diagnostic tools, relies on a single bispecific antibody which recognizes both PRA and PRB, in certain contexts, despite the (1) variety of human breast cancers and (2) the existence of PRA and PRB epitopes which may escape detection with the single bispecific anti-PR antibody.

In the presence of a bound progestin ligand, such as progesterone, the PR is phosphorylated at specific sites, dimerizes, forms a complex with a number of different cellular elements (e.g., p300 and the steroid receptor coactivator), and binds to specific DNA sequences known as progesterone responsive elements (PREs) to initiate DNA transcription into RNA.

These complex biological modulations of the PR are known to be abnormal in cancers where for example abnormal PR phosphorylation could take place via alternate biological pathways. It is therefore plausible that PRA and PRB are differentially altered in breast cancer and other cancers, and that a bispecific antibody directed to PR isoforms modified in a specific manner would lose its sensitivity and specificity for PRA or a PRB. Thus, standard diagnostic methods could fail to detect PRA and PRB resulting in an incorrect or false classification of TNBC.

In contrast to normal breast tissue, the level of PRA and PRB expression is not present in an equal ratio. Relative expression of the PR isotypes separated by immunoblots appears to be prognostic in malignant tumors (Hopp et al., Clin Cancer Res. 2004 Apr. 15; 10(8):2751-60)). Tumors can be heterogeneous with respect to PRA and PRB expression—different cells in the same tumor can express different PR subtypes. (Mote et. al., Ernst Schering Foundation Proceedings, Vol 1. pp 77-107, 2008). These malignant cells can also differentially express ERα (Zukiwski et. al., Proc ASCO, abstract #118076, 2013) but the overlapping expression of ERα and PR in individual cancer cells is low. Publications referred to herein are incorporated by reference in their entirety.

Each PR isoform has a different physiological role. The PR has a central DNA binding domain and a carboxy-terminus ligand binding domain. PRB has an additional 164 amino acids at its N-terminus when compared to PRA. The two isoforms are otherwise identical. (Breast Cancer Res 2002, 4:187-190). The isoforms are phosphorylated under various conditions as illustrated in FIG. 1.

Standard testing for the steroid receptors has demonstrated that primary BCs are usually ERα positive and PR positive. PR is routinely assayed with a bispecific antibody which has been selected to detect both PRA and PRB. By this method, 20% of ERα-positive BCs are PR negative while about 5% of PR-positive BCs are ERα negative. Triple negative BCs are defined as ERα, PR, and HER2/neu negative. In principle by standard IHC testing techniques, TNBCs are both PRA and PRB negative.

The accuracy of the triple negative breast cancer phenotype determination depends, in part, on the ability of the antibody to detect both PRA and PRB expression. There are biological elements which point to the fact that PRB might be uniquely altered in breast cancer, and that the routine detection of both PRA and PRB with a single antibody could miss the altered expression of PRA or PRB. A true triple negative BC phenotype might escape detection with the use of one single Ab to detect both PRA and PRB epitopes, depending on the sensitivity/specificity of the antibody, which could result in providing incorrect information to patients.

### SUMMARY

One aspect provides methods of identifying a progesterone receptor positive tumor by obtaining a tissue sample suspected of being tumorigenic or cancerous from a patient, identifying progesterone receptor positive cells in the tissue sample using at least a first antibody for detecting PRA and at least a second antibody for detecting PRB, and determining that the tissue sample is progesterone receptor positive if the presence of PRA or the presence of PRB is detected.

Other aspects provide methods of identifying a triple negative phenotype by obtaining a BC tissue sample suspected of being tumorigenic or cancerous from a patient, identifying progesterone receptor positive cells in the tissue sample using at least a first antibody for detecting PRA and at least a second antibody for detecting PRB, detecting the presence of the estrogen receptor alpha (ER α) using at least one antibody for detecting the estrogen receptor, detecting the presence of HER2/neu using at least one validated test for determining the presence of HER2/neu (e.g., immunohistochemistry, in situ hybridization), and determining that the tissue sample is a triple negative phenotype if the presence of PRA, PRB, the ER α, and HER2/neu is not detected.

Yet other aspects provide methods of inhibiting the growth of a tumor susceptible to growth inhibition by anti-progestins by obtaining a tissue sample suspected of being tumorigenic or cancerous from a patient, identifying progesterone receptor positive cells in the tissue sample using at least a first antibody for detecting PRA and at least a second antibody for detecting PRB, determining that the tissue sample is progesterone receptor positive if the presence of PRA or the presence of PRB is detected progesterone receptor positive if the presence of PRA or the presence of PRB is detected, and administering anti-endocrine therapy (e.g., anti-progestins) if the tissue sample is determined to be progesterone receptor positive.

Further aspects provide a system for classifying a tumor as progesterone positive, comprising a tissue sample and at least a first antibody or antibody binding fragment capable of detecting PRA and at least a second antibody or antibody binding fragment capable of detecting PRB wherein the at least a first antibody or antibody binding fragment and at least a second antibody or antibody binding fragment is used to determine if the tumor is PRA positive or PRB positive or both PRA positive and PRB positive.

Another aspect provides a kit for identifying a progesterone positive tumor comprising at least a first antibody or antibody binding fragment capable of detecting PRA and at least a second antibody or antibody binding fragment capable of detecting PRB.

### BRIEF DESCRIPTION OF THE DRAWINGS

The feature and nature of the present disclosure will become more apparent from the detailed description set forth below when taken in conjunction with the accompanying drawings in which reference characters identify corresponding items.

FIG. 1 shows PRA and PRB isoform receptor domains and post-translational modifications; and

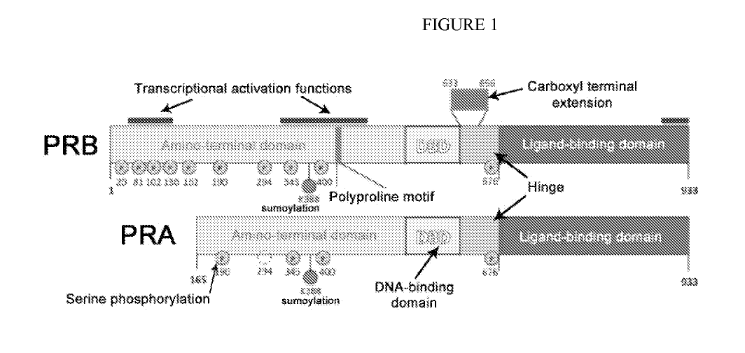

FIG. 2 shows dual staining of PRA and PRB with estrogen receptor alpha in breast cancer tissue.

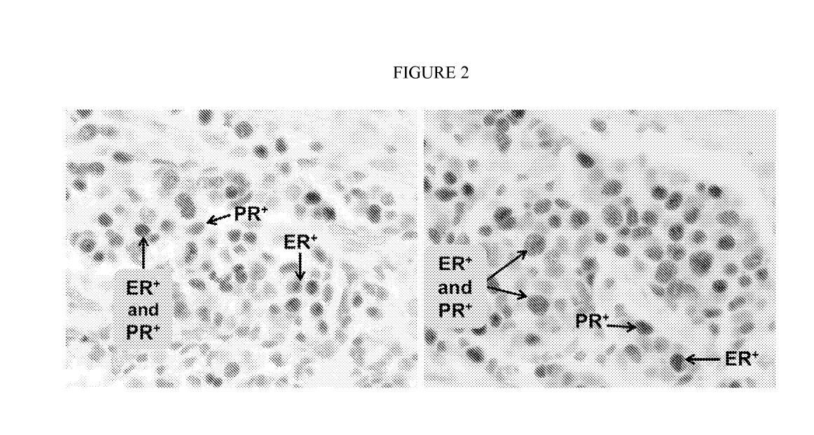

### DETAILED DESCRIPTION

The disclosed methods and devices below may be described both generally as well as specifically. It should be noted that when the description is specific to an aspect, that aspect should in no way limit the scope of the aspects described herein. All references cited herein are hereby incorporated by reference in their entirety.

In one aspect, two isotype-specific PR Abs are used to fully characterize PR status. In this example, 520 archived BC specimens with available clinical and laboratory testing data were obtained from Oscar Lambret Cancer Center, Lille, France. HER2 status was previously determined. IHC was performed using anti-ERα, anti-PRA and anti-PRB Abs. In this example, ERα and PR positive tumors are defined as tumors having ≧1% stained tumor cells using the indicated antibodies.

IHC was performed on 3-4 μm sections of archival BC tissues. Samples were prepared for immunostaining using standard methodology (e.g., http://www:dako.com/us/ar38/p105760/prod_products.htm). A standard hematoxylin was generated for IHC. Negative controls were obtained by substitution of the primary antibodies with isotype control mouse IgG1 or with antibody diluent alone (wash buffer negative control)

520 archived breast cancer tumors were obtained and retested for ERα, PRA and PRB. 71 cases were determined to be ERα negative by retesting and HER2 negative from patient records. The 71 Era negative and HER2 negative cases were also tested with the 2 PR bispecific antibodies, and 50 of these cases had adequate tissue available for complete testing with the multiple antibodies.

TABLE 1
Antibodies used for IHC

| Antibody | Clone | Concentration |
|---|---|---|
| PRA | Novacastra 16 | 1:500/1:100 |
| PRB | Novacastra SANS27 | 1:500/1:100 |
| Bispecific | PgR636 | 1:50 |
| Bispecific | 1A6 | 1:100 |

Of the bispecific antibodies tested, Pg636 had qualitatively better staining and more BC were identified as PR positive than with the 1A6 antibody (36 vs 32%).

The detection of PRA and PRB relative to Pg636 or 1A6 is presented in Tables 2 and 3. 27/50 cases were found to be “true” triple negative, using all PR antibodies. In this example, the bispecific antibodies provided the same result as the individual PRA and PRB antibodies. 13/50 cases were PR positive by all antibodies tested. However, 10/50 cases demonstrated discordant results for either PRA, PRB, Pg636 or 1A6.

TABLE 2
Cross-tabulation of PR Positive Samples using Bispecific Antibodies
vs. PRA Isoform-specific Antibody in 50 BC Specimens

| | PRA Novacastra 16 | | PRA Novacastra 16 | | | |
|---|---|---|---|---|---|---|
| | Negative, n (%) | | Positive, n (%) | | Total | |
| Pg636 Negative | 30 | (60) | 2 | (4) | 32 | (64) |
| Pg636 Positive | 3 | (6) | 15 | (30) | 18 | (36) |
| Total | 33 | (66) | 17 | (34) | 50 | (100) |
| 1A6 Negative | 30 | (60) | 4 | (8) | 34 | (68) |
| 1A6 Positive | 3 | (6) | 13 | (26) | 16 | (32) |
| Total | 33 | (66) | 17 | (34) | 50 | (100) |

As shown below, the percentage of PRA negative and PRA positive samples detected with the PRA isotype-specific antibody (PRA Novacastra 16) are shown in parenthesis, while the number of PR negative and positive samples detected with the PG636 and 1A6 bispecific antibodies are shown without parenthesis.

TABLE 3
Cross-tabulation of PR Positivity Using Bispecific Antibodies
vs. PRB Isoform-specific Antibody in 50 BC Specimens

| | PRB Novacastra | | PRB Novacastra | | | |
|---|---|---|---|---|---|---|
| | SANS 27 | | SANS 27 | | | |
| | Negative, n (%) | | Positive, n (%) | | Total | |
| Pg636 Negative | 28 | (56) | 4 | (8) | 32 | (64) |
| Pg636 Positive | 5 | (10) | 13 | (26) | 18 | (36) |
| Total | 33 | (66) | 17 | (340 | 50 | (100) |
| 1A6 Negative | 29 | (58) | 5 | (10) | 24 | (68) |
| 1A6 Positive | 4 | (8) | 12 | (24) | 16 | (32) |
| Total | 33 | (66) | 17 | (34) | 50 | (100) |

As shown below, the percentage of PRB negative and PRB positive samples detected with the PRA isotype-specific antibody (PRA Novacastra SANS 27) are shown in parenthesis, while the number of PR negative and positive samples detected with the PG636 and 1A6 bispecific antibody bispecific antibodies are shown without parenthesis.

TABLE 4
Discordant Data From Testing With Different PR Antibodies

| P636 | 1A6 | PRA | PRB |
|---|---|---|---|
| Neg | Neg | Neg | Pos |
| Neg | Neg | Neg | Pos |
| Neg | Neg | Neg | Pos |
| Neg | Neg | Pos | Pos |
| Neg | Pos | Pos | Neg |
| [symbol] | Neg | [symbol] | [symbol] |
| [symbol] | Neg | Pos | [symbol] |
| [symbol] | Pos | [symbol] | [symbol] |
| [symbol] | Pos | [symbol] | [symbol] |
| [symbol] | Pos | Pos | [symbol] |

Table 4 illustrates the 10 cases with discordant data from testing with the different PR antibodies. As shown below in Table 4, samples that are classified as PR negative using the bispecific antibodies (Pg636 and 1A6) are classified as PRB positive, PRA positive, or both PRB positive and PRA positive using the isotype specific antibodies directed to PRA and to PRB.

Of the 50 samples tested, 37 could potentially be classified as triple negative depending on the PR antibody used for the IHC testing. Ten cases had discordant PR results. In one aspect, use of PRA and PRB isotype-specific antibodies may reveal those BC cases that are PR positive but were previously identified as a triple negative phenotype using bispecific antibodies.

In one aspect, the information captured by PRB (isotype-specific) and Pg636 (bispecific) is not concordant. For example, when Pg636 indicated a sample was PR negative, the sample was PRB positive in 4/5 cases. When Pg636 indicated a sample was PR positive, the sample was PRB negative in 5/5 cases. Thus, the diagnostic information provided by the bispecific antibodies was not correct.

In another aspect, the use of isotype specific PR antibodies identifies PR positive BC tissue samples that are classified as PR negative and thus have a triple negative phenotype using PR bispecific antibodies. Therefore, the use of PRA and PRB isotype-specific antibodies is an important method of detecting PR-positive tumors that may be undetected using bispecific antibodies. Thus, patients with previously undetected PR-positive tissue samples may be amenable to treatment with anti-endocrine therapies, including anti-progestins.

TABLE 5
PR and ER Status of Patient Population

| | | | |
|---|---|---|---|
| | PR Status | PR Status | |
| ER Status | Negative | Positive | Total |

| | | | | | | |
|---|---|---|---|---|---|---|
| Negative | 53 | (12.05%) | 28 | (6.36%) | 81 | (18.41) |
| Positive | 41 | (9.32%) | 318 | (72.27) | 359 | (81.59) |
| Total | 94 | (21.36) | 346 | (78.64) | 440 | (100%) |

As shown in Table 5, the PR and ER status of 520 patients from the Oscar Lambret Cancer Center, Lille, France 440 was determined. A sample is considered PR positive if it is either PRA or PRB positive. Out of 359 ER positive cases, 41 (9%) were PR negative −11% less than the 20% expected with the use of a bispecific antibody. The 11% represents samples that were determined to be PR negative using bispecific antibodies but are actually PR positive as determined through the use of PRA and PRB antibodies. The 11% are, therefore, from patients that may be amendable to treatment with anti-endocrine therapies, including anti-progestins. Using bispecific antibodies, these patients would not have been identified as PR positive and therefore would have incomplete information regarding treatment options.

In one aspect, methods of administering an anti-progestin to a patient susceptible to treatment with an anti-progestin are provided. In this aspect, breast tissue samples are obtained from a patient having breast cancer, progesterone receptor positive cells in the tissue sample are identified using at least a first antibody for detecting PRA and at least a second antibody for detecting PRB, the tissue sample is identified as progesterone receptor positive if the presence of PRA or the presence of PRB is detected. The presence of the ERα is determined using at least one antibody for detecting the estrogen receptor and an anti-progestin is administered to the patient if the tissue sample is determined to be progesterone receptor positive and estrogen receptor positive.

As used herein, the term “antibody” or “antibodies”, unless otherwise indicated, refers to an immunoglobulin that binds a specific antigen and includes, but is not limited to, polyclonal, monoclonal, chimeric, or humanized antibodies, Fab fragments or any other fragment that binds to at least a portion of the antigen. “Antibodies” included any class of antibodies or immunoglobulins including, but not limited to, IgG, IgA, IgM, IgD, IgE, and secreted immunoglobulins in any configuration (e.g., two identical heavy chains and two light chains, single chain antibodies, two chain antibodies).

Aspects described herein provide methods of identifying a progesterone receptor positive tumor by obtaining a tissue sample suspected of being tumorigenic or cancerous from a patient, identifying progesterone receptor positive cells in the tissue sample using at least a first antibody for detecting PRA and at least a second antibody for detecting PRB, and determining that the tissue sample is progesterone receptor positive if the presence of PRA or the presence of PRB is detected.

Further aspects provide methods of identifying a triple negative phenotype breast cancer tumor by obtaining a tissue sample suspected of being tumorigenic or cancerous from a patient, identifying progesterone receptor positive cells in the tissue sample using at least a first antibody for detecting PRA and at least a second antibody for detecting PRB, detecting the presence of the estrogen receptors using at least one antibody for detecting the estrogen receptor α; detecting the presence of HER2/neu using at least one validated test for determining the presence of HER2/neu, and determining that the tissue sample is a triple negative phenotype if the presence of PRA, PRB, the ERα; estrogen receptor, and HER2/neu is not detected.

Yet additional aspects provide systems for classifying a tumor as progesterone positive, comprising a tissue sample and at least a first antibody capable of detecting PRA and at least a second antibody capable of detecting PRB wherein the at least a first antibody and at least a second antibody is used to determine if the tumor is PRA positive or PRB positive or both PRA positive and PRB positive.

Kits for identifying a progesterone positive tumor comprising at least a first antibody capable of detecting PRA and at least a second antibody capable of detecting PRB are also provided. In this aspect, the kits can further comprise reagents needed to detect PRA and PRB.

In another aspect, the tissue sample used for aspects described herein are obtained from breast tissue. In another aspect, the first antibody and second antibody are isotype specific antibodies (e.g., PRA Novacastra 16, PRB Novacastra 16).

Further aspects provide methods of administering an anti-progestin to a patient susceptible to treatment with anti-endocrine therapy by obtaining a BC sample from a patient having breast cancer, identifying progesterone receptor positive cells in the tissue sample using at least a first antibody for detecting PRA and at least a second antibody for detecting PRB, determining that the tissue sample is progesterone receptor positive if the presence of PRA or the presence of PRB is detected, detecting the presence of the estrogen receptor α using at least one antibody for detecting the estrogen receptor, and administering anti-endocrine therapy to the patient if the tissue sample is determined to be progesterone receptor positive and estrogen receptor α positive. In this aspect, anti-endocrine therapy comprises an anti-progestin.

Further aspects provide methods of inhibiting the growth of a tumor susceptible to growth inhibition by anti-progestins by obtaining a tissue sample suspected of being tumorigenic or cancerous from a patient, identifying progesterone receptor positive cells in the tissue sample using at least a first antibody for detecting PRA and at least a second antibody for detecting PRB, determining that the tissue sample is progesterone receptor positive if the presence of PRA or the presence of PRB is detected, and administering an anti-progestin to the patient if the tissue sample is determined to be progesterone receptor positive.

In another aspect, the anti-progestin is selected from the group consisting onapristone, lonaprisan, mifepristone, PF-02413873, telapristone, lilopristone, ORG2058, apoprisnil, ulipristal, ZM172406, ZM150271, ZM172405 and aglepristone.

Onapristone, (e.g., (8S,11R,13R,14S,17S)-11-[4-(dimethylamino)phenyl]-17-hydroxy-17-(3-hydroxypropyl)-13-methyl-1,2,6,7,8,11,12,14,15,16-decahydrocyclopenta[a]phenanthren-3-one) has the following chemical structure:

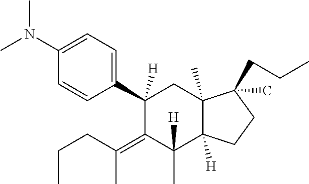

Other anti-progestins include: progestational 3-(6,6-ethylene-17B-hydroxy-3-oxo-17A-pregna-4-ene-17A-YL)propionic acid G-lactones, 3-(6,6-ethylene-17.beta.-hydroxy-3-oxo-17.alpha.-pregna-4-ene-17.alpha.-y-1)propionic acid .gamma.-lactone and the following:

Mifepristone (10S,11 S,14S,15 S,17R)-17-[4-(dimethylamino)phenyl]-14-hydroxy-15-methyl-14-(prop-1-yn-1-yl)tetracyclo[8.7.0.0̂ {2,7}0.0̂ {11,15}]heptadeca-1,6-dien-5-one

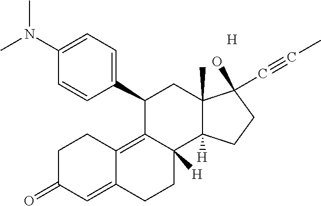

Lilopristone (11-beta,17-beta,17(z))-ropenyl); estra-4,9-dien-3-one,11-(4-(dimethylamino)phenyl)-17-hydroxy-17-(3-hydroxy-1-p; 11β-[4-(Dimethylamino)phenyl]-17β-hydroxy-17-[(Z)-3-hydroxy-1-propenyl]estra-4,9-dien-3-one

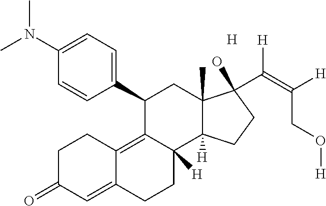

ORG2058 (8R,9S,10R,13S,14S,16R,17S)-16-ethyl-17-(2-hydroxyacetyl)-13-methyl-2,6,7,8,9,10,11,12,14,15,16,17-dodecahydro-1H-cyclopenta[a]phenanthren-3-one

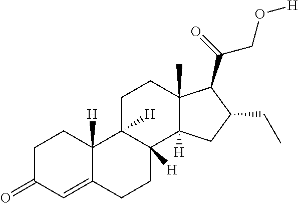

Lonaprisan (8S,11R,13S,14S,17S)-11-(4-acetylphenyl)-17-hydroxy-13-methyl-17-(1,1,2,2,2-pentafluoroethyl)-1,2,6,7,8,11,12,14,15,16-decahydrocyclopenta[a]phenanthren-3-one

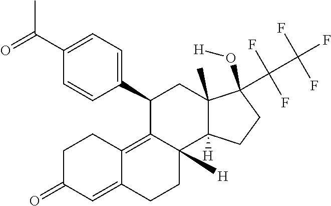 [SMILES: CC(=O)c1ccc([C@H]2C[C@@]3(C)[C@@H](CC[C@@]3(O)C(F)(F)C(F)(F)F)[C@@H]3CCC4=CC(=O)CCC4=C32)cc1]

Asoprisnil (8S,11R,13 S,14S,17S)-11-[4-[(E)-hydroxyiminomethyl]phenyl]-17-methoxy-17-(methoxymethyl)-13-methyl-1,2,6,7,8,11,12,14,15,16-decahydrocyclopenta[a]phenanthren-3-one

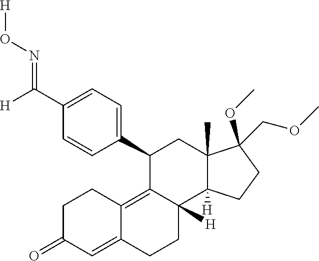 [SMILES: COC[C@]1(OC)CC[C@H]2[C@@H]3CCC4=CC(=O)CCC4=C3[C@@H](c3ccc(/C=N/O)cc3)C[C@@]21C]

Ulipristal (8S,11R,13S,14S,17R)-17-acetyl-11-[4-(dimethylamino)phenyl]-17-hydroxy-13-methyl-1,2,6,7,8,11,12,14,15,16-decahydro cyclopenta[a]phenanthren-3-one

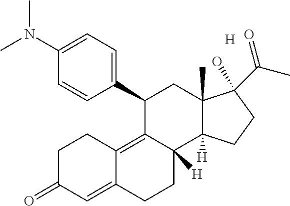

PF-2413873 4-[3-Cyclopropyl-1-(mesylmethyl)-5-methyl-1H-pyrazol-4-yl]oxy,-2,6-dimethylbenzonitrile

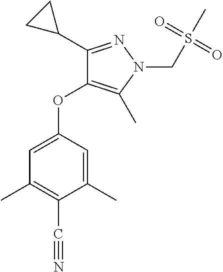 [SMILES: Cc1cc(Oc2c(C3CC3)nn(CS(C)(=O)=O)c2C)cc(C)c1C#N]

Aglepristone (8S,11R,13 S,14S,17R)-11-(4-dimethylaminophenyl)-17-hydroxy-13-methyl-17-[(Z)-prop-1-enyl]-1,2,6,7,8,11,12,14,15,16-decahydrocyclopenta[a]phenanthren-3-one:

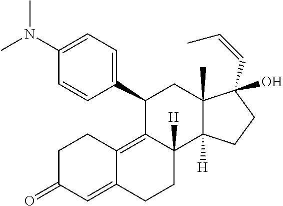

Additional anti-progestins include the following:

ZM172406—(R)—N-(3-chloro-4-cyanophenyl-3,3,3-trifluoro-2-hydroxy-2-methylpropanamide:

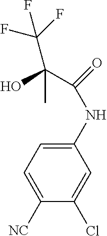 [SMILES: C[C@@](O)(C(=O)Nc1ccc(C#N)c(Cl)c1)C(F)(F)F]

ZM172405—(S)—N-(3-chloro-4-cyanophenyl)-3,3,3-trifluoro-2-hydroxy-2-methylpropanamide:

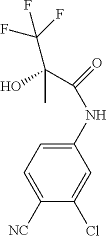 [SMILES: C[C@](O)(C(=O)Nc1ccc(C#N)c(Cl)c1)C(F)(F)F]

ZM150271—N-(3-chloro-4-cyanophenyl)-3,3,3,-trifluoro-2-hydroxy-2-methylpropanamide:

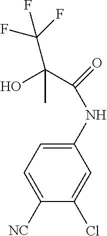 [SMILES: CC(O)(C(=O)Nc1ccc(C#N)c(Cl)c1)C(F)(F)F]

In a further aspect, the anti-progestin can be administered to a patient in an amount from about 10 mg to about 200 mg per day. Optionally, an anti-tumor compounds (e.g., everolimus, trastuzumab, TM1-D, anti-HER2 drugs, bevacizumab, paclitaxel, docetaxel, taxanes, doxorubicin, liposomal doxorubicin, pegylated liposomal doxorubicin, anthracyclines, anthracenediones, carboplatin, cisplatin, 5-FU, gemcitabine, cyclophosphamide, anti-estrogen, selective estrogen receptor modulators, aromatase inhibitors, and anti-androgens) may also be administered to the patient concurrently, before, or after treatment with the anti-progestin.

### EXAMPLES

### Example 1

A semi-quantitative PR/APR IHC assay is designed to identify progesterone receptor (PR) and activated progesterone receptor (APR, as evident by aggregate PR staining) expression in neoplastic and surrounding normal tissues that are formalin-fixed and paraffin-embedded (FFPE) for histological evaluation. The proposed assay will:

1) detect both the PR-A and PR-B isoforms located in the cytoplasm and nuclei of PR-expressing cells and

2) distinguish between the activated and non-activated forms of these receptors by assessment of subnuclear localization.

An IHC technique has been developed utilizing FFPE primary endometrioid cancer specimens to assess and analyze the subnuclear distribution of PR. Two anti-PR antibodies corresponding to PR-A and PR-B will be used in this assay; Novocastra clone 16 and Novocastra clone SAN27 respectively. Both the technique and reagents form the basis of the proposed IVD.

Detection and analysis of PR/APR will be performed on three separate microtome tumor sections obtained by surgical excision or biopsy from each individual patient. One microtome section for each of the two anti-PR antibodies (PR-A and PR-B) and an isotype control antibody.

Both of these anti-PR antibodies bind to their respective PR targets in both activated and non-activated forms in the nuclei of both normal and neoplastic cells. It is necessary to deploy antibodies for the two isoforms because the expression and nuclear distribution of PR-A and PR-B vary and are not consistent in cancer tissue specimens including endometrioid cancer.

The three individual slides stained with the specific PR antibody will be examined and analyzed by light microscopy to semi-qualitatively identify the presence and relative expression of the following:

1) PR in the tumor specimen;

Interpretation of PR status will be based on independent review of multiple fields of each of the PR-A and PR-B stained slides at low magnification for overall staining using the conventional 0, +1, +2, +3 scale with +2 or greater being reported to the ordering physician as PRpos; and

2) APR (Activated Progesterone Receptor) in tumor specimens;

Interpretation of APR status will be based on independent review of multiple fields of each of the PR-A and PR-B stained slides at high magnification (at least 60× objective with NA of at least 0.9) for the percent of PRpos cells, which exhibit the nuclear focal pattern consistent with APR with the % determined in a prospective clinical trial being reported to the ordering physician as APRpos.

TABLE 6
PR/APR IHC Protocol on BOND-III

| | | Temp | Incubation Time |
|---|---|---|---|
| Step Number | Reagent Name | (° C.) | (minutes) |
| 1 | Peroxide Block | Ambient | 5 |
| 2 | BOND Wash Solution | Ambient | 0 |
| 3 | BOND Wash Solution | Ambient | 0 |
| 4 | BOND Wash Solution | Ambient | 0 |
| 5 | PRIMARY | Ambient | 30 |
| 6 | BOND Wash Solution | Ambient | 0 |
| 7 | BOND Wash Solution | Ambient | 0 |
| 8 | BOND Wash Solution | Ambient | 0 |
| 9 | Post Primary | Ambient | 8 |
| 10 | BOND Wash Solution | Ambient | 2 |
| 11 | BOND Wash Solution | Ambient | 2 |
| 12 | BOND Wash Solution | Ambient | 2 |
| 13 | Polymer | Ambient | 8 |
| 14 | BOND Wash Solution | Ambient | 2 |
| 15 | BOND Wash Solution | Ambient | 2 |
| 16 | Deionized Water | Ambient | 0 |
| 17 | Mixed DAB Refine | Ambient | 0 |
| 18 | Mixed DAB Refine | Ambient | 10 |
| 19 | Deionized Water | Ambient | 0 |
| 20 | Deionized Water | Ambient | 0 |
| 21 | Deionized Water | Ambient | 0 |
| 22 | Hematoxylin | Ambient | 5 |
| 23 | Deionized Water | Ambient | 0 |
| 24 | BOND Wash Solution | Ambient | 0 |
| 25 | Deionized Water | Ambient | 0 |

### Example 2

### Separate Reagents

(1) deparaffinizing agent in buffer, (2) wash buffer, (3) counterstain for structural identification (e.g., hematoxylin), (4) Novocastra Clone 16 in buffer for PRA detection, (5) Novocastra Clone SANS27 in buffer for PRB detection, (6) Buffer containing secondary antibodies to the FAB fragments of each detection antibody conjugated to an enzyme (e.g., horse radish peroxidase) (7) Substrate solution for the enzyme-conjugated antibody, and (8) Quenching solution to arrest enzyme activity.

Apparatus:

Automated, advanced slide staining device.

Protocol: (all Steps to be Conducted at Optimized Times and Temperatures)

(1) Two (2) 3-4 μm slides will be made from FFPE blocks by microtome, (2) Each will be mounted in the automated, advanced slide stainer, (3) Deparaffinizing step using agent, (4) Wash step, (5) Incubation with counterstain (6) Wash step, (7) Incubation with one primary antibody per slide (e.g., Clone 16 and SANS27), (8) Wash step, (9) Incubation with secondary antibody-enzyme conjugate, (10) Wash step, (11) Incubation with detection substrate (12) Wash step (13) Analysis, (14) The two anti-PR-stained slides, along with one hematoxylin/eosin stained slide, from each specimen will be interpreted by at least two qualified pathologists for PR expression and APR status, (15) Slides will be scanned at high resolution and images made available for consultation.

Not every element described herein is required. Indeed, a person of skill in the art will find numerous additional uses of and variations to the methods described herein, which the inventors intend to be limited only by the claims.

What is claimed as new and desired to be protected by Letters Patent of the United States is:

1. A method of identifying a progesterone receptor positive tumor, comprising:
obtaining a tissue sample suspected of being tumorigenic or cancerous from a patient;
identifying progesterone receptor positive cells in the tissue sample using at least a first antibody for detecting PRA and at least a second antibody for detecting PRB; and
determining that the tissue sample is progesterone receptor positive if the presence of PRA or the presence of PRB is detected.

2. The method of claim 1, wherein the tissue sample is obtained from breast tissue.

3. The method of claim 1, wherein the first antibody and second antibody are isotype specific antibodies.

4. The method of claim 3, where in the first antibody is PRA Novacastra 16 and the second antibody is PRB Novacastra 16.

5. A method of identifying a triple negative phenotype breast cancer tumor, comprising:
obtaining a tissue sample suspected of being tumorigenic or cancerous from a patient;
identifying progesterone receptor positive cells in the tissue sample using at least a first antibody for detecting PRA and at least a second antibody for detecting PRB;
detecting the presence of the estrogen receptor α using at least one antibody for detecting the estrogen receptor α; detecting the presence of HER2/neu using at least one validated test for determining the presence of HER2/neu; and
determining that the tissue sample is a triple negative phenotype if the presence of PRA, PRB, the ERα; estrogen receptor, and HER2/neu is not detected.

6. The method of claim 5, wherein the tissue sample is obtained from breast tissue.

7. The method of claim 5, wherein the first antibody and second antibody are isotype specific antibodies.

8. The method of claim 7, where in the first antibody is PRA Novacastra 16 and the second antibody is PRB Novacastra 16.

9. A system for classifying a tumor as progesterone positive, comprising a tissue sample and at least a first antibody capable of detecting PRA and at least a second antibody capable of detecting PRB wherein the at least a first antibody and at least a second antibody is used to determine if the tumor is PRA positive or PRB positive or both PRA positive and PRB positive.

10. The method of claim 9, wherein the tissue sample is obtained from breast tissue.

11. The method of claim 9, wherein the first antibody and second antibody are isotype specific antibodies.

12. The method of claim 11, where in the first antibody is PRA Novacastra 16 and the second antibody is PRB Novacastra 16.

13. A method of administering an anti-progestin to a patient susceptible to treatment with anti-endocrine therapy comprising:
obtaining a BC sample from a patient having breast cancer;
identifying progesterone receptor positive cells in the tissue sample using at least a first antibody for detecting PRA and at least a second antibody for detecting PRB;
determining that the tissue sample is progesterone receptor positive if the presence of PRA or the presence of PRB is detected;
detecting the presence of the estrogen receptor α using at least one antibody for detecting the estrogen receptor; and
administering anti-endocrine therapy to the patient if the tissue sample is determined to be progesterone receptor positive and estrogen receptor α positive.

14. The method of claim 13, wherein the anti-endocrine therapy comprises an anti-progestin.

15. The method of claim 13, wherein the tissue sample is obtained from breast tissue.

16. The method of claim 13, wherein the first antibody and second antibody are isotype specific antibodies.

17. The method of claim 16, where in the first antibody is PRA Novacastra 16 and the second antibody is PRB Novacastra 16.

18. The method of claim 14, wherein the anti-progestin is selected from the group consisting onapristone, lonaprisan, mifepristone, PF-02413873, telapristone, lilopristone, ORG2058, apoprisnil, ulipristal, ZM172406, ZM150271, ZM172405 and aglepristone.

19. The method of claim 14, further comprising administering an anti-tumor compound selected from the group consisting of everolimus, trastuzumab, TM1-D, anti-HER2 drugs, bevacizumab, paclitaxel, docetaxel, taxanes, doxorubicin, liposomal doxorubicin, pegylated liposomal doxorubicin, anthracyclines, anthracenediones, carboplatin, cisplatin, 5-FU, gemcitabine, cyclophosphamide, anti-estrogen, selective estrogen receptor modulators, aromatase inhibitors, and anti-androgens.

20. A method of inhibiting the growth of a tumor susceptible to growth inhibition by anti-progestins, comprising:
obtaining a tissue sample suspected of being tumorigenic or cancerous from a patient;
identifying progesterone receptor positive cells in the tissue sample using at least a first antibody for detecting PRA and at least a second antibody for detecting PRB;
determining that the tissue sample is progesterone receptor positive if the presence of PRA or the presence of PRB is detected; and
administering an anti-progestin to the patient if the tissue sample is determined to be progesterone receptor positive.
